(19) United States
(12) Plant Patent Application Publication
(10) Pub. No.: US 2012/0331597 P1
(43) Pub. Date: Dec. 27, 2012

(54) Grapevine 'IFG Five'

(50) ;

(75) Inventor: David Cain, Bakersfield, CA (US)

(73) Assignee: International Fruit Genetics, LLC, Bakersfield, CA (US)

(21) Appl. No.: 13/134,948

(22) Filed: Jun. 22, 2011

(51) Int. Cl. A01H 5/00

(52) U.S. Cl. PLT/205

(57) ABSTRACT

This invention is a new and distinct grapevine variety named ‘IFG Five’ which is characterized by producing extremely large purple to black, firm, elongated oval seeded grapes borne on large clusters. The fruit ripen and are harvestable in mid-August.

### LATIN NAME OF THE GENUS AND SPECIES CLAIMED

Vitis vinifera

### VARIETY DENOMINATION

‘IFG Five’

### BACKGROUND OF THE INVENTION

The new and distinct grapevine described and claimed herein originated from a hand pollinated cross of the (Redglobe variety U.S. Plant Pat. No. 4,787 expired) and the Summer Royal variety (non-patented) performed in May of 2001. The seeds were subsequently germinated and the resulting plants were planted in the field in April 2002. The present variety of grapevine was selected as a single plant in August of 2003 and was first asexually propagated by hardwood cuttings in December of 2003. The resulting propagules were planted during April 2004 near Delano, Kern County, Calif. and were found to reproduce true-to-type through at least two generations of asexual reproduction.

### BRIEF SUMMARY OF THE INVENTION

The new grapevine ‘IFG Five’ is characterized by producing extremely large, elongated oval seeded purplish to black berries borne on large clusters. The vine is very productive with short spur pruning and the fruit ripen 7 to 10 days before its maternal parent variety Redglobe. Berries have firm texture and excellent eating quality. It attains full black color with difficulty and may exhibit green or red coloration around the pedicle under some conditions. Application of the growth regulator ethrel allows full coloration.

The new grape vine differs from its parent the Redglobe variety by ripening 7 to 10 days earlier and by having larger more elongated berries which are black in color as opposed to the rounder red berries of Redglobe. It differs from its pollen parent by producing much larger seeded berries as opposed to the smaller seedless berries of the Summer Royal variety. To the inventor's knowledge, the known variety to which the new grapevine variety is most similar is the ‘Black Globe’ (U.S. Plant Pat. No. 17,875 P3). ‘IFG Five’ can be distinguished from Black Globe by ripening earlier (mid to late August as opposed to early to mid September for Black Globe), by having a more elongated ovate shape as opposed to the obovoid shape of ‘Black Globe’. ‘IFG Five’ is more productive than the ‘Black Globe’ variety and can be spur pruned and still be highly productive while the ‘Black Globe’ is only medium productive and requires more costly cane pruning to maximize bunch counts. Mature clusters of ‘IFG Five’ are somewhat larger than ‘Black Globe’ and the peduncle is longer.

### BRIEF DESCRIPTION OF THE FIGURE

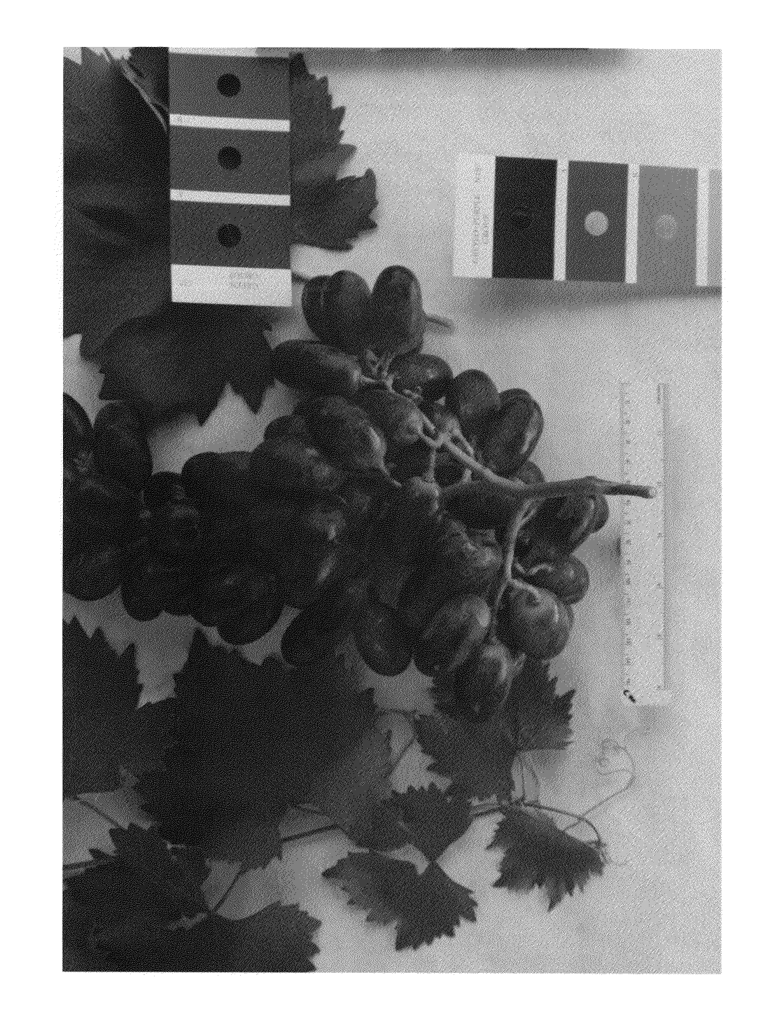

The accompanying photographic illustration in FIG. 1 illustrates in full color ‘IFG Five’. The colors are as nearly true as is reasonably possible in a color representation of this type.

### DETAILED BOTANICAL DESCRIPTION OF THE INVENTION

Throughout this specification, color names beginning with a small letter signify that the name of that color, as used in common speech, is aptly descriptive. Color names beginning with a capital letter designate values based upon R.H.S. Colour Chart, published by The Royal Horticultural Society, London, England.

Throughout this specification subjective description values conform to those set forth by the International Plant Genetic Resources Institute publication ‘Descriptors for Grape’ (Vitis spp.) (1983) which was developed in collaboration with the Office International de la Vigne et du Vin (OIV) and the International Union for the Protection of New Varieties of Plants (UPOV).

The descriptive matter which follows pertains to ‘IFG Five’ plants grown in the vicinity of Delano, Kern County, Calif. during 2009, and is believed to apply to plants of the variety grown under similar conditions of soil and climate elsewhere:
Vine:
   General.—Size — Large Vigor — Medium Density of foliage — Medium Productivity — Very productive Root stock — Own root Training method — Typically spur pruned leaving 2 bud spurs.
   Trunk.—Trunk diameter of 4-year-old vines at 30 cm above the soil line — 6.2 cm Shape — Stocky Straps — Very long — Continuous Surface texture — Medium Inner bark color — Greyed-orange 165A, 166A, 177B, 177C.
Shoots:
   Young shoot.—Form of tip — Wide open Distribution of anthocyanin coloration of tip — Absent Intensity of anthocyanin coloration of tip — Absent Density of prostrate hairs of tip — Sparse Density of erect hairs of tip — Absent Color — 144A.
   Woody shoot (mature canes).—Shape — Medium thick Internode length — Medium, About 10.3 cm Width at node — About 1.0 cm Cross section — Circular to Elliptic Surface — Striate Main color — Yellowish brown; 166A, B, C, 164A, B, 176A, C Density of erect hairs of nodes — None or very sparse Density of erect hairs on internodes — None or very sparse Growth of axillary shoots — Strong; Approximately 34.8 cm.
   Flowering shoot.—Vigor during flowering — Medium to Strong Attitude during flowering on shoots not tied — Erect to Semi-erect Color — dorsal side of internodes — Green with Red stripes Color — ventral side of internodes — Green Color — dorsal side of nodes — Green with Red stripes Color — ventral side of nodes — Green Density of prostrate hairs of nodes — None or Very sparse Density of erect hairs of nodes — None Density of prostrate hairs on internode — None Density of erect hairs on internode — None Anthocyanin coloration of buds — Absent.
   Tendrils.—Distribution on the shoot (at full flowering) — Discontinuous Length of Tendril — Long; About 26.5 cm Thickness — Medium Color — Green; 145A Form — Bifurcated and Trifurcated Number of consecutive tendrils — 2.
Leaves:
   Young leaves.—Color of upper surface of first four distal unfolded leaves — Green with bronze spots Average intensity of anthocyanin coloration of fourth distal leaves prior to flowering — Medium Density of prostrate hairs between veins (lower surface) — Absent Density of prostrate hairs on veins (lower surface) — Very sparse Density of erect hairs between veins (lower surface) — Absent Density of erect hairs on veins (lower surface) — Very sparse.
   Mature leaves.—Average length — About 16.2 cm Average width — About 19.7 cm Mature leaf size — Medium Shape of blade — Wedge-shaped Number of lobes — 5 Anthocyanin coloration of main veins on upper side of blade — Weak — Medium Mature leaf profile — V-shaped Blistering surface of blade upper surface — Weak Leaf blade tip — In the plane of the leaf Undulation of margin — Slight — Medium Thickness — Medium Undulation of blade between main and lateral veins — Absent Shape of teeth — Mixture of both sides straight and both sides convex Length of teeth — Medium Ratio length/width of teeth — Small Shape of upper lateral sinuses — Closed — Lobes slightly overlapping Depth of upper lateral sinuses — Medium Shape of base of upper leaf sinuses — V-shaped General shape petiole sinus — Lobes slightly overlapping Shape of base of petiole sinus — U shaped Tooth at petiole sinus — Absent Density of prostrate hairs between veins on lower surface of blade — Absent Density if erect hairs between veins on lower surface of blade — Absent Density of prostrate hairs on main veins on lower surface of blade — Very sparse Density of erect hairs on main veins on lower surface of blade — Sparse Density of prostrate hairs on main veins on upper surface of blade — Very sparse Density of erect hairs on main veins on upper surface of blade — None Autumn coloration of leaves — Red-Purple; 59A, 60A, Yellow-Green; 152 D, C, 153B, C.
   Upper surface.—Color — Yellow-Green; 147A Anthocyanin coloration of main veins — Weak — Medium Surface appearance — Semi-glossy — Dull Blistering surface of blade — Weak.
   Lower surface.—Color — Green; 146A Glossiness — Weak Surface texture — Smooth Surface appearance — Dull.
   Petiole.—Length — About 12.8 cm Length of petiole compared to middle vein — Slightly shorter Density of prostrate hairs on petiole — None Density of erect hairs on petiole — None.
   Buds.—Bud fruitfulness — Basal: Mostly fruitful Position of first fruitful shoot on previous season cane — 1^{st} to 2^{nd} node Time of bud burst — Early, Mar. 3, 2010.
Flowers:
   General.—Flower sex — Hermaphrodite Length of first inflorescence — Long; about 23.0 cm long by 13.0 cm wide Position of first flowering and fruiting node — 4^{th}-5^{th} node (current season growth) Number of inflorescence per flowering shoot — 1.1 to 2 Time of bloom — Midseason as compared with similar varieties in the growing area of Delano, Calif. Date of full bloom — Mar. 18, 2010.
Fruit:
   General.—Ripening period — Medium; Approximately Aug. 23, 2010 Use — Fresh market Keeping quality — Good Resistance to — Insects: Average, typical of Vitis vinifera species; Diseases: Average, typical of Vitis vinifera species Refractometer test — solid-sugar: About 19.0 Brix Brix/acid — About 38.8 Titratable acidity — About 0.49 Juice pH — About 3.82.
   Cluster.—Mature cluster length (peduncle excluded) — About 29.6 cm Mature cluster width — About 19.7 cm Mature cluster weight — About 1422 g Bunch density — Medium Number of berries — About 139 Form — Circular to Cylindrical.
   Peduncle.—Lignification of peduncle — Medium Length of peduncle — Approximately 6.3 cm.
   Berry.—Uniformity of size — Uniform Single berry weight — About 12.8 g naturally; to about 14.9 g when treated with gibberellic acid or vine girdling Shape — Ovate Seeds — Seeded Cross section — Circular Berry dimensions — longitudinal axis: About 33 mm. horizontal axis: About 25 mm Berry firmness — Firm Particular flavor — Neutral Bloom (cuticular wax) — Medium Berry separation from pedicel — Difficult Skin color (without bloom) — Dark red-violet — blue-black; about 187A.
   Skin.13 Thickness — Medium Texture — Medium Reticulation — Present Tenacity — Tenacious to flesh.

What is claimed:

1. A new and distinct variety of grapevine as herein illustrated and described.
